(19) United States
(12) Patent Application Publication
(10) Pub. No.: US 2025/0050023 A1
(43) Pub. Date: Feb. 13, 2025

(54) A sub-assembly of a medicament delivery device

(71) Applicant: SHL Medical AG, Zug (CH)

(72) Inventors: Shinshiuan Chiou, Zug (CH); Ming-Ting Yin, Taoyuan City (TW)

(21) Appl. No.: 18/718,500

(22) PCT Filed: Nov. 30, 2022
(86) PCT No.: PCT/EP2022/083770

§ 371 Date: Jun. 11, 2024

(30) Foreign Application Priority Data
Dec. 14, 2021 (EP) 21214271.5

(51) Int. Cl. A61M 5/20; A61M 5/32

(52) U.S. Cl. CPC A61M 5/2033; A61M 2005/2006; A61M 2005/3267

(57) ABSTRACT

The present disclosure provides a sub-assembly of a medicament delivery device, the sub-assembly comprising: a housing extending along a longitudinal axis between a proximal end and a distal end, a plunger rod arranged within the housing; wherein the plunger rod comprises a plunger rod body and a ledge extending from the plunger rod body in a direction transverse to the longitudinal axis; a biasing member configured to bias the plunger rod relative to the housing in the direction of the longitudinal axis; and a plunger rod holder axially immovable relative to the housing; wherein the ledge of the plunger rod defines two opposite surfaces facing towards the distal end and the proximal end of the housing respectively; and wherein one of the two opposite surfaces is configured to engage with the biasing member and the other one of the two opposite surfaces is configured to releasably engage with the plunger rod holder.

### CROSS-REFERENCE TO RELATED APPLICATIONS

The present application is a U.S. National Phase Application pursuant to 35 U.S.C. § 371 of International Application No. PCT/EP2022/083770 filed Nov. 30, 2022, which claims priority to European Patent Application No. 21214271.5 filed Dec. 14, 2021. The entire disclosure contents of these applications are herewith incorporated by reference into the present application.

### TECHNICAL FIELD

The present disclosure generally relates to a sub-assembly of a medicament delivery device, and particularly to a sub-assembly comprises a plunger rod comprising a plunger rod body and a ledge extending from the plunger rod body.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone.

It is an advantage when the medicament delivery devices for the self-administration comprise automatic functions, so that even if users are without professional training or are in an emergency situation, the users can easily and properly use the medicament delivery devices.

Even though many of the devices on the market, as well as the ones described above, have their respective advantages, there is still room for improvements.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device, the sub-assembly comprising: a housing extending along a longitudinal axis between a proximal end and a distal end, a plunger rod arranged within the housing; the plunger rod comprises a plunger rod body and a ledge extending from the plunger rod body in a direction transverse to the longitudinal axis; a biasing member configured to bias the plunger rod relative to the housing in the direction of the longitudinal axis; and a plunger rod holder axially immovable relative to the housing. The ledge of the plunger rod defines two opposite surfaces facing towards the distal end and the proximal end of the housing respectively. One of the two opposite surfaces is configured to engage with the biasing member and the other one of the two opposite surfaces is configured to releasably engage with the plunger rod holder.

Preferably, according to another embodiment, the housing comprises a proximally directed surface; the ledge defines a distally directed surface and a proximally directed surface; the biasing member extends along the longitudinal axis between a proximal end and a distal end; the proximal end of the biasing member is adjacent to the distally directed surface of the ledge of the plunger rod, and the distal end of the biasing member is adjacent to the proximally directed surface of the housing.

Preferably, according to another embodiment, the biasing member is a compression spring surrounding at least a part of the plunger rod body.

Preferably, according to another embodiment, the plunger rod holder comprises a distally directed surface; and the plunger rod holder is movable relative to the plunger rod between a first position where the distally directed surface aligns with and faces towards the proximally directed surface of the ledge of the plunger rod and a second position where the distally directed surface is transverse offset to the proximally directed surface of the ledge of the plunger rod.

Preferably, according to another embodiment, the plunger rod holder comprises a flexible arm that can flex from the first position to the second position; the flexible arm extends from a wall of the housing, and the distally directed surface of the plunger rod holder is on a protrusion extending from the flexible arm in the direction transverse to the longitudinal axis.

Alternatively, according to another embodiment, the plunger rod holder is rotatable around the longitudinal axis relative to the housing from the first position to the second position; the plunger rod holder comprises a protrusion extending towards the plunger rod body; and the distally directed surface of the plunger rod holder is on the protrusion.

Preferably, the plunger rod holder comprises a cylindrical or semi-cylindrical holder body; and the protrusion of the plunger rod holder extends from a wall of the holder body.

Preferably, according to another embodiment, the sub-assembly comprises an actuator comprising an actuator surface; the plunger rod holder comprises an actuated surface facing towards the actuator surface; wherein at least one of the actuator surface and the actuated surface is bevelled relative to the longitudinal axis; and the actuator surface is configured to move along the actuated surface with a movement of the actuator relative to the plunger rod holder in the direction of the longitudinal axis, thereby moving the plunger rod holder from the first position to the second position.

Preferably, according to another embodiment, the actuator surface is a distally directed surface; the actuated surface is a proximally directed surface and wherein the actuator surface is configured to move along the actuated surface with a movement of the actuator relative to the plunger rod holder towards the distal end of the housing, thereby moving the plunger rod holder from the first position to the second position.

Preferably, according to another embodiment, the actuated surface is bevelled between a proximal end to a distal end; and the actuator is axially movable relative to the plunger rod holder from a proximal position where the actuator surface is adjacent to the proximal end of the actuated surface to a distal position where the actuator surface is adjacent to the distal end of the actuated surface.

Preferably, according to another embodiment, the sub-assembly comprises a delivery member cover axially movable relative to the housing; the delivery member cover comprises a tubular section configured to surround a medicament delivery member of the medicament delivery device; the delivery member cover comprises an arm extending from the tubular section towards the distal end of the housing, and the actuator extends from the arm of the delivery cover towards the longitudinal axis.

Preferably, according to another embodiment, the sub-assembly comprises an outer shell extending along the longitudinal axis between a proximal end and a distal end; the outer shell is configured to receive a medicament container of the medicament delivery device; the housing is axially immovable arranged within the outer shell.

Preferably, according to another embodiment, the plunger rod comprises an engaging member; the engaging member is closer to the distal end of the outer shell than the ledge of the plunger rod; the housing comprises a counter engaging member engaged with the engaging member of the plunger rod; one of the engaging member and the counter engaging member is arranged on a flexible arm; the sub-assembly comprises safety member removably attached to the distal end of the outer shell; a cut-out arranged at the distal end of the outer shell; and the safety member comprises a support wall protruding into the outer shell through the cut-out of the outer shell and configured to block the flexible arm from flexing.

Preferably, according to another embodiment, the housing comprises a distal opening; the counter engaging member of the housing is a distally directed surface extending from the distal end of the housing into the distal opening; the plunger rod comprises a flexible arm protruding from the distal opening of the housing; the engaging member of the plunger rod is a proximally directed surface on the flexible arm; the proximally directed surface adjacent to the distally directed surface of the housing; and the flexible arm is arranged between the support wall of the safety member and an edge of the distal opening of the housing in the direction transverse to the longitudinal axis.

Alternatively, according to another embodiment, the engaging member of the plunger rod is a recess or cut-out is arranged in a sidewall of the plunger rod body; the housing comprises a flexible arm extending from a distally directed wall of the housing towards a proximally directed wall of the outer shell; the counter engaging member of the housing is a protrusion extending from the flexible arm; the protrusion of the flexible arm protrudes into the recess or cut-out of the plunger rod body, and the support wall of the safety member encloses an outer surface of the flexible arm of the housing.

Preferably, according to another embodiment, the cut-out of the outer shell is arranged in the proximally directed wall of the outer shell.

Preferably, according to another embodiment, the tubular section of the delivery member cover is telescopic relative to the proximal end of the outer shell.

Preferably, according to another embodiment, the sub-assembly is used in a medicament delivery device comprising a multiple-chamber medicament container arranged within the carrier.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery member is an injection needle or a spray nozzle.

Preferably, according to another embodiment, the medicament container is syringe or a cartridge.

Generally, the medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the medicament) and a pharmaceutically acceptable carrier. Pharmaceutical formulations comprising a drug as listed herein (or a pharmaceutically acceptable salt of the medicament) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device comprising a sub-assembly of the invention.

FIG. 2 schematically shows an exploded view of the sub-assembly of FIG. 1.

FIG. 3 schematically shows a perspective view of a plunger rod and a biasing member of the sub-assembly of FIG. 1.

FIGS. 4A-4B schematically show a perspective view of a housing and a plunger rod holder of the sub-assembly of FIG. 1.

FIG. 5 schematically shows a perspective view of a delivery member cover of the sub-assembly of FIG. 1.

FIG. 6 schematically shows a perspective view of the delivery member cover of FIG. 5 and the plunger rod holder of FIGS. 4A-4B.

FIG. 7 schematically shows a perspective view of the plunger rod and the biasing member of FIG. 3 and the housing and the plunger rod holder of FIGS. 4A-4B.

FIG. 8 schematically shows a perspective view of the plunger rod and the biasing member of FIG. 3, the plunger rod holder of FIGS. 4A-4B and the delivery member cover of FIG. 5.

FIG. 9 schematically shows a perspective view of the plunger rod of FIG. 3, and the plunger rod holder of FIGS. 4A-4B when observed in a direction along the longitudinal axis.

FIG. 10 schematically shows a perspective view of a medicament delivery device comprising a sub-assembly of the invention in another embodiment.

FIG. 11 schematically shows a perspective view of the housing of the sub-assembly of FIG. 10.

FIG. 12 schematically shows a perspective view of a safety member of the sub-assembly of FIG. 10.

FIG. 13 schematically shows a cross-section view of the sub-assembly of FIG. 10.

### DETAILED DESCRIPTION

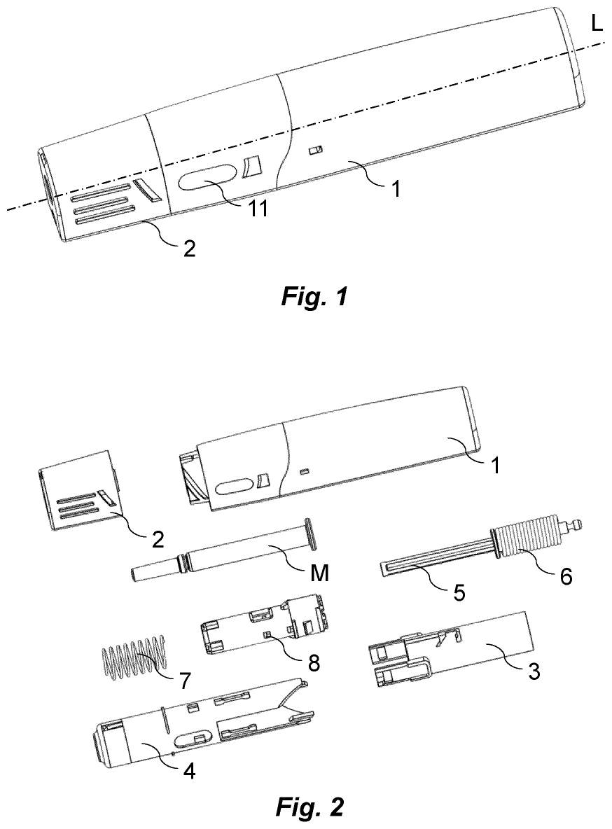

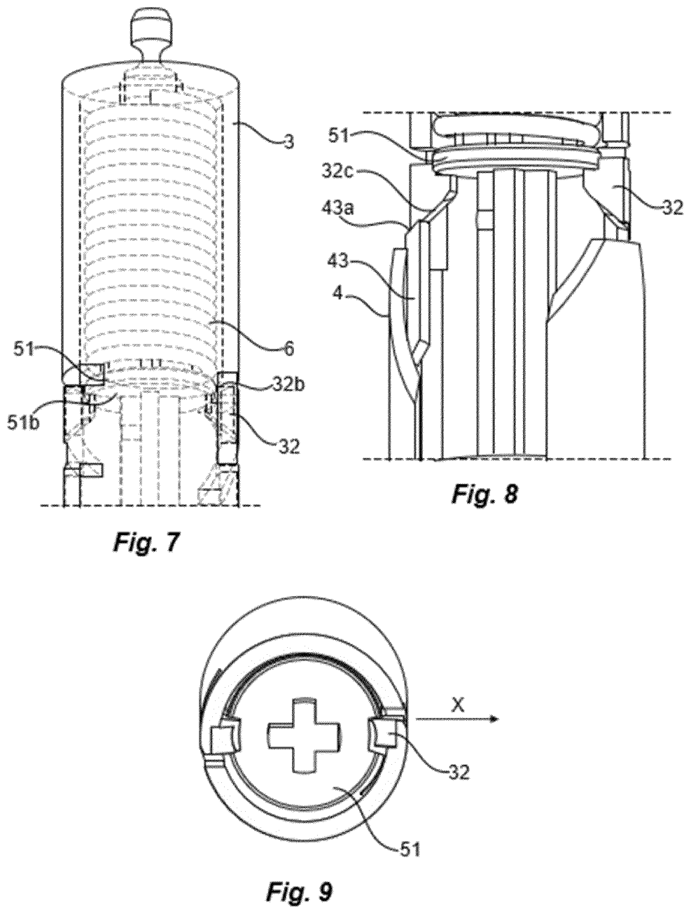

FIGS. 1-13 illustrate a sub-assembly of a medicament delivery device, the sub-assembly comprising: a housing 3 extending along a longitudinal axis L between a proximal end and a distal end, a plunger rod 5 arranged within the housing 3, a biasing member 6 configured to bias the plunger rod 5 relative to the housing 3 in the direction of the longitudinal axis L, and a plunger rod holder 32 axially immovable relative to the housing 3. As shown in FIG. 4A, the housing 3 comprises a tubular body 30 configured to receive the plunger rod 5 and the biasing member 6.

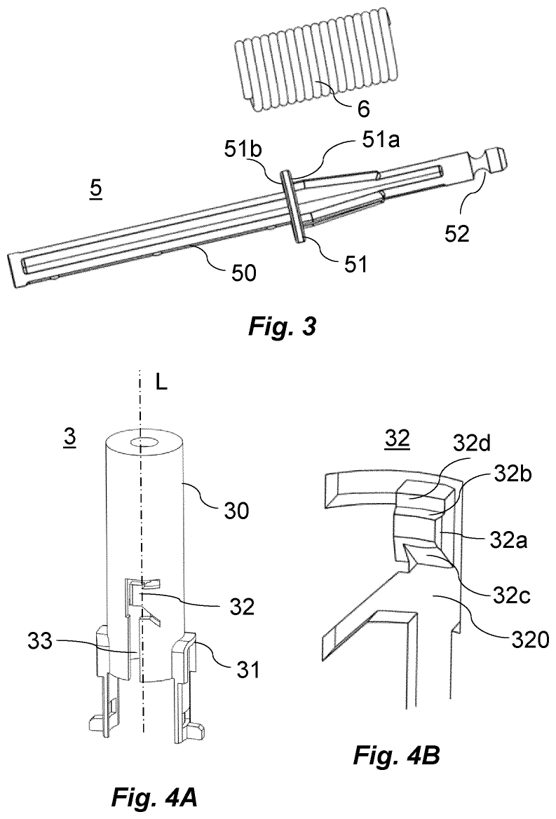

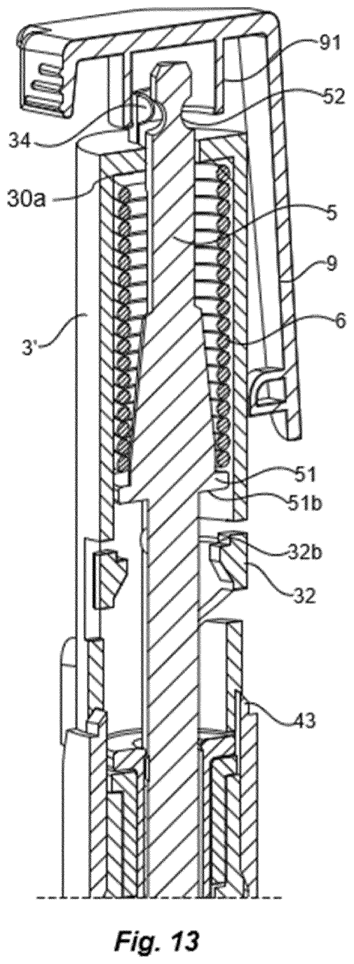

As shown in FIG. 3, the plunger rod 5 comprises a plunger rod body 50 and a ledge 51 extending from the plunger rod body 50 in a direction transverse to the longitudinal axis L. The plunger rod body 50 extends in the direction of the longitudinal axis L. In this example, the ledge 51 of the plunger rod 5 is an annular ledge 51. Alternatively, the ledge is an arm or semi-circular ledge extending partially around the plunger rod body. The ledge 51 defines two opposite surfaces 51a, 51b facing towards the distal end and the proximal end of the housing 3 respectively. One of the two opposite surfaces is configured to engage with the biasing member 6 and the other one of the two opposite surfaces is configured to releasably engage with the plunger rod holder 32. The two opposite surfaces are a distally directed surface 51a and a proximally directed surface 51b. In the example, shown in FIG. 3, the distally directed surface 51a is configured to engage with the biasing member and the proximally directed surface 51b is configured to releasably engage with the plunger rod holder 32. In this example, the biasing member 6 extends along the longitudinal axis L between a proximal end and a distal end. The proximal end of the biasing member 6 is adjacent to the distally directed surface 51a of the ledge 51 of the plunger rod 5 and the distal end of the biasing member 3 is adjacent to a proximally directed surface 30a of the housing 3, as shown in FIG. 13. In a preferred example, the biasing member 3 is a compression spring 3 surrounding at least a part of the plunger rod body 50, as shown in FIG. 13.

The plunger rod holder 32 comprises a distally directed surface 32b configured to releasably engage with the proximally directed surface 51b of the ledge 51 of the plunger rod 5. The plunger rod holder 32 is movable relative to the plunger rod 5 between a first position where the distally directed surface 32b aligns with and faces towards the proximally directed surface 51b of the ledge 51 of the plunger rod 5 and a second position where the distally directed surface 32b is transverse offset to the proximally directed surface 51b of the ledge 51 of the plunger rod 5. Therefore, when the plunger rod holder 32 is in the second position, the plunger rod 5 is no longer blocked by the distally directed surface 32b of the plunger rod holder 32 from moving towards the proximal end of the housing, so that the plunger rod 5 can be moved in the direction of the longitudinal axis relative to the housing 3 under by the biasing member 6.

In the example, shown in FIGS. 4A-4B, the plunger rod holder 32 comprises a flexible arm 320 that can flex from the first position to the second position. The flexible arm 320 extends from a wall of the housing 3. Preferably, the flexible arm extends from a wall of the tubular body 30 of the housing 3. The distally directed surface 32b of the plunger rod holder 32 is on a protrusion 32a extending from the flexible arm 320 in the direction transverse to the longitudinal axis L. In this example, when the plunger rod holder 32 is in the second position, the distally directed surface 32b is radially offset to the proximally directed surface 51b of the ledge 51 of the plunger rod 5. Optionally, the plunger rod holder 32 comprises a support surface 32d facing towards the longitudinal axis L. The support surface 32d is configured to support the ledge 51 of the plunger rod 5 from a side of the ledge 51.

Alternatively, the plunger rod holder is rotatable around the longitudinal axis relative to the housing from the first position to the second position. In this example, the plunger rod holder comprises a protrusion extending towards the plunger rod body. The distally directed surface of the plunger rod holder is on the protrusion. In this example, when the plunger rod holder is in the second position, the distally directed surface is circumferentially offset from the proximally directed surface of the ledge of the plunger rod. In this example, the plunger rod holder comprises a cylindrical or semi-cylindrical holder body. The protrusion of the plunger rod holder extends from a wall of the holder body.

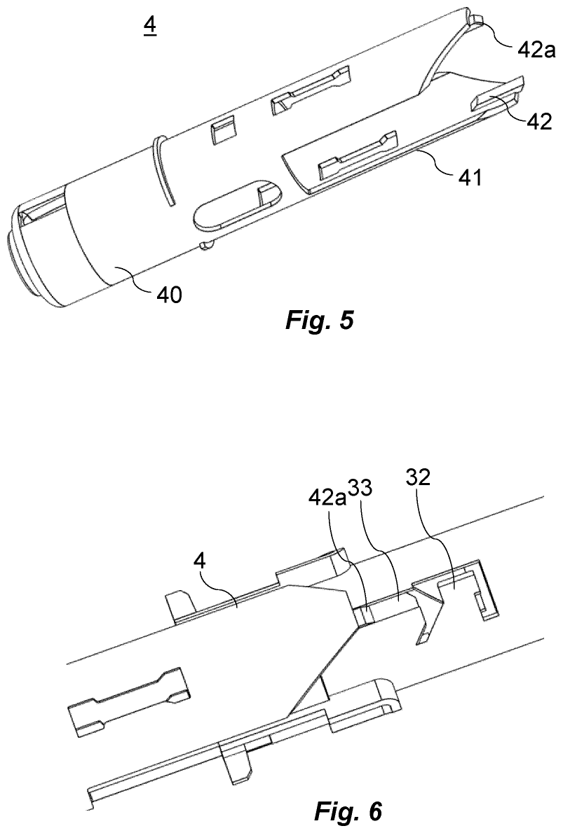

In the example shown in FIG. 2 and FIG. 5, the sub-assembly comprises an actuator 42 comprising an actuator surface 42a. The plunger rod holder 32 comprises an actuated surface 32c facing towards the actuator surface 42a, as shown in FIG. 8. The actuator 42 is configured to move in the direction of the longitudinal axis relative to the housing 3 to move the plunger rod holder 32 from the first position to the second position. At least one of the actuator surface and the actuated surface is bevelled relative to the longitudinal axis L, so that when the actuator surface of the actuator axially moves along the actuated surface of the plunger rod holder, the plunger rod holder is moved from the first position to the second position. In a preferred example, both actuator surface 42a and the actuated surface 32c are bevelled relative to the longitudinal axis L, as shown in FIG. 8.

In a preferred example, the actuator surface 42a is a distally directed surface and the actuated surface 32c is a proximally directed surface, as shown in FIG. 8. In this example, the actuator surface 42a is configured to move along the actuated surface 32c with a movement of the actuator 42 relative to the plunger rod holder 32 towards the distal end of the housing 3, thereby moving the plunger rod holder 32 from the first position to the second position. In this example, the sub-assembly comprises a delivery member cover 4 axially movable relative to the housing 3, as shown in FIG. 2 and FIG. 5. The delivery member cover 4 comprises a tubular section 40 configured to surround a medicament delivery member of the medicament delivery device. The delivery member cover 4 comprises an arm 41 extending from the tubular section towards the distal end of the housing 3. The actuator 42 extends from the arm 41 of the delivery cover 4 towards the longitudinal axis L, as shown in FIG. 5. In a preferred example, the actuated surface 32c is bevelled between a proximal end to a distal end. The actuator 42 is axially movable relative to the plunger rod holder 32 from a proximal position where the actuator surface 42a is adjacent to the proximal end of the actuated surface 32c to a distal position where the actuator surface 42a is adjacent to the distal end of the actuated surface 32c.

In one example where the plunger rod holder 32 comprises the flexible arm 320, the actuated surface 32c is on the protrusion 32a, as shown in FIG. 4B. In this example, when the actuator 42 axially moves in the direction of the longitudinal axis L relative to the plunger rod holder 32, the actuator surface 42a moves in contact with the actuated surface 32c, then moves along the actuated surface 32c, thereby moving the flexible arm 320 flexes radially relative to the plunger rod in the direction transverse to the longitudinal axis. In the example shown in FIG. 9, the flexible arm 320 flexes radially outward relative to the plunger rod (shown as the X direction in FIG. 9). The distally directed surface 32b of the plunger rod holder 32 thereby is radially offset to the proximally directed surface 51b of the ledge 51 of the plunger rod 5. It should be noted that the flexible arm can also be configured to flex radially inward relative to the plunger rod to move into the second position, dependent on the bevelling direction of the actuated surface. This example is preferred when the ledge of the plunger rod is an arm instead of an annular ledge.

Alternatively, in one example where the plunger rod holder is rotatable around the longitudinal axis L relative to the housing, the actuated surface is arranged on the holder body, so that the axial movement of the actuator causes the actuator surface to move along the actuated surface and thereby rotating the plunger rod holder from the first position to the second position. In this example, the distally directed surface of the plunger rod holder thereby is circumferentially offset to the proximally directed surface of the ledge of the plunger rod.

In a preferred example, the housing 3 comprises a slot 33 in the wall of the tubular body 30. The slot 33 extends from a proximal end of the tubular body to the plunger rod holder 32. The actuator 42 is configured to move along the slot 33, as shown in FIG. 6, towards the plunger rod holder 32.

In a preferred example, the sub-assembly comprises a delivery member cover biasing member 7 configured to move the delivery member cover in the proximal direction relative to the housing 3. The sub-assembly optionally comprises a container carrier 8 configured to receive a medicament container M, e.g. a syringe or a cartridge. In a preferred example, the container carrier 8 is axially fixed to the housing 3, e.g. by a snap-fit engagement with a fastener 31 of the housing 3. In this example, the delivery member cover biasing member 7 is positioned between the container carrier 8 and the tubular section 40 of the delivery member cover 4. For example, the delivery member cover biasing member 7 is a compression spring 7 adjacent to a distally directed surface of the container carrier 8 at a distal end of the compression spring, and adjacent to a proximally directed surface of the tubular section 40 of the delivery member cover 4. Therefore, the tubular section 40 of the delivery member cover 4 is moved to a position where the tubular section covers the medicament delivery member by the delivery member cover biasing member 7. When a user wants to carry out a medicament delivery operation, the user will move the delivery member cover 4 in the distal direction relative to the housing 3, so that the medicament delivery member can be exposed. This distal movement of the delivery member cover 4 causes the actuator 42 to axially move relative to the plunger rod holder 32 from the proximal position to the distal position, and thereby the plunger rod holder 32 is moved by the actuator 42 from the first position to the second position, as mentioned above.

It should be noted that the actuator can also be attached to a button at the distal end of the housing or position on a sidewall of the tubular body of the housing. If the button is configured to be moved from a distal position to a proximal position for triggering a medicament delivery operation, instead of the example mentioned above, in this example, the actuator surface 42a is a proximally directed surface and the actuated surface 32c is a distally directed surface.

In another example, the plunger rod 5 is axially movable relative to the housing 3′ from a distal position where the proximally directed surface 51b of the ledge 51 of the plunger rod 5 is spaced apart from the distally directed surface 32b of the plunger rod holder 32 to a proximal position where the proximally directed surface 51b of the ledge 51 of the plunger rod 5 is adjacent to the distally directed surface 32b of the plunger rod holder 32. In this example, the plunger rod 5 comprises an engaging member 52. In a preferred example, the engaging member 52 is closer to the distal end of the plunger rod 5 than the ledge 51, as shown in FIG. 3. The housing 3′ comprises a counter engaging member 34 engaged with the engaging member 52 of the plunger rod 5, as shown in FIG. 13. One of the engaging member and the counter engaging member is arranged on a flexible arm; in a preferred example, the counter engaging member 34 comprises an arm extending from the distal end of the housing 3′ in the distal direction of the housing 3′. In a preferred example, the counter engaging member 34 is a distally directed surface extending from the distal end of the housing 3′. In this example, the counter engaging member is a protrusion extending from the arm in the direction transverse to the longitudinal axis L and thereby defining the distally directed surface. In this example, the engaging member 52 of the plunger rod is formed by a recess or a cut-out 52 in the plunger rod body 50. When the engaging member 34 and the counter engaging member are engaged together, the protrusion of the counter engaging member 34 is positioned within the recess or cut-out 52 of the plunger rod 5, as shown in FIG. 11 and FIG. 13. The sub-assembly comprises a removable support wall 91 enclosing the arm of the counter engaging member 34 of the housing 3′. For example, the removable support wall 91 can be formed by a ring or a tubular component that is removably attached to the housing 3′. Therefore, when the support wall encloses the arm of the counter engaging member 34 of the housing 3′, the plunger rod 5 is held in the distal position by the engagement between the engaging member 52 and the counter engaging member 34, as shown in FIG. 13. The interface formed between the engaging member 52 and the counter engaging member 34 is bevelled, so that when the support wall 91 is removed from the housing 3′, the biasing member 6 moves the plunger rod 5 to deflect the arm of the counter engaging member 34, thereby disengaging the engaging member 52 of the plunger rod 5 from the counter engaging member 34 of the housing 3′ and moving the plunger rod 5 to the proximal position.

Alternatively, the housing comprises a distal opening, and the counter engaging member of the housing is a distally directed surface extending from the distal end of the housing into the distal opening. In this example, the plunger rod comprises a flexible arm protruding from the distal opening of the housing. In this example, the engaging member of the plunger rod is a proximally directed surface on the flexible arm.

In this example, the medicament delivery device with the sub-assembly can provide two medicament delivery shots (by releasing the plunger rod twice). For example, the first shot can be a priming shot for expelling any contained air within the medicament container M, or a mixing shot for mixing multiple contained substances within multiple chambers of the medicament container. The second shot is configured to deliver the contained medicament to a medicament delivery site of a patient.

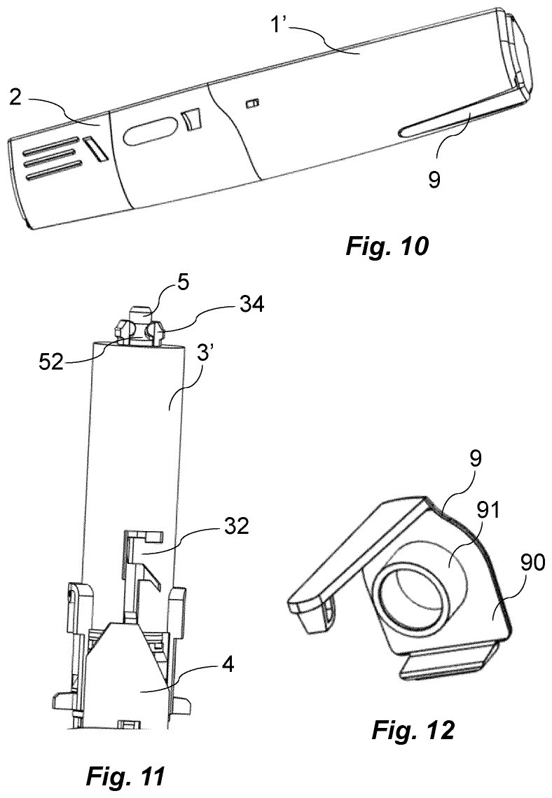

In a preferred example, the sub-assembly comprises an outer shell 1; 1′ extending along the longitudinal axis L between a proximal end and a distal end, as shown in FIG. 1, FIG. 2 and FIG. 10. The outer shell 1; 1′ is configured to receive the medicament container M of the medicament delivery device. The housing 3; 3′ is axially immovably arranged within the outer shell 1; 1′. In this example, the sub-assembly comprises a safety member 9 removably attached to the distal end of the outer shell 1′, as shown in FIG. 10 and FIG. 12. In this example, a cut-out is arranged at the distal end of the outer shell 1′. In this example, the safety member 9 comprises a support wall 91 protruding into the outer shell 1′ through the cut-out of the outer shell 1′ and configured to block the flexible arm 34 from flexing. In a preferred example, the safety member 9 comprises a user-accessible body 90. The support wall 91 extends from a proximal side of the user-accessible body 90 into the cut-out of the distal end of the outer shell 1′. A part of the user-accessible body 90 is configured to be attached on the outer shell 1′, as shown in FIG. 10, so that the user can easily remove the safety member 9 from the outer shell 1′. In a preferred example, the cut-out of the outer shell 1′ is arranged in the proximally directed wall of the outer shell 1′.

In one example where the sub-assembly comprises the delivery member cover 4, the tubular section 40 of the delivery member cover 4 is telescopic relative to the proximal end of the outer shell 1′. In another example, the sub-assembly comprises a cap 2 configured to cover the proximal end of the delivery member cover. In a preferred example, the cap 2 is removably attached to the proximal end of the outer shell 1′, so that an unintentional movement of the delivery member cover 4 towards the distal end of the housing 3′ can be prevented.

The inventive concept has mainly been described above with reference to a few examples. However, as is readily appreciated by a person skilled in the art, other embodiments than the ones disclosed above are equally possible within the scope of the inventive concept, as defined by the appended claims.

1-16. (cancelled)

17. A sub-assembly of a medicament delivery device, the sub-assembly comprising:
a housing extending along a longitudinal axis between a proximal end and a distal end;
a plunger rod arranged within the housing, wherein the plunger rod comprises a plunger rod body and a ledge extending from the plunger rod body in a direction transverse to the longitudinal axis;
a biasing member configured to bias the plunger rod relative to the housing in the direction of the longitudinal axis; and
a plunger rod holder axially immovable relative to the housing, wherein the ledge of the plunger rod defines two opposite surfaces facing towards the distal end and the proximal end of the housing respectively, and wherein one of the two opposite surfaces is configured to engage with the biasing member and the other one of the two opposite surfaces is configured to releasably engage with the plunger rod holder.

18. The sub-assembly according to claim 17, wherein the housing comprises a proximally directed surface, wherein the ledge defines a distally directed surface and a proximally directed surface, wherein the biasing member extends along the longitudinal axis between a proximal end and a distal end, wherein the proximal end of the biasing member is adjacent to the distally directed surface of the ledge of the plunger rod, and wherein the distal end of the biasing member is adjacent to the proximally directed surface of the housing.

19. The sub-assembly according to claim 17, wherein the biasing member is a compression spring surrounding at least a part of the plunger rod body.

20. The sub-assembly according to claim 17, wherein the plunger rod holder comprises a distally directed surface, and wherein the plunger rod holder is movable relative to the plunger rod between a first position where the distally directed surface aligns with and faces towards the proximally directed surface of the ledge of the plunger rod and a second position where the distally directed surface is transverse offset to the proximally directed surface of the ledge of the plunger rod.

21. The sub-assembly according to claim 20, wherein the plunger rod holder comprises a flexible arm that can flex from the first position to the second position, wherein the flexible arm extends from a wall of the housing, and wherein the distally directed surface of the plunger rod holder is on a protrusion extending from the flexible arm in the direction transverse to the longitudinal axis.

22. The sub-assembly according to claim 20, wherein the sub-assembly comprises an actuator comprising an actuator surface, wherein the plunger rod holder comprises an actuated surface facing towards the actuator surface, wherein at least one of the actuator surface and the actuated surface is bevelled relative to the longitudinal axis, and wherein the actuator surface is configured to move along the actuated surface with a movement of the actuator relative to the plunger rod holder in the direction of the longitudinal axis, thereby moving the plunger rod holder from the first position to the second position.

23. The sub-assembly according to claim 22, wherein the actuator surface is a distally directed surface, wherein the actuated surface is a proximally directed surface, and wherein the actuator surface is configured to move along the actuated surface with a movement of the actuator relative to the plunger rod holder towards the distal end of the housing, thereby moving the plunger rod holder from the first position to the second position.

24. The sub-assembly according to claim 23, wherein the actuated surface is bevelled between a proximal end to a distal end, and wherein the actuator is axially movable relative to the plunger rod holder from a proximal position where the actuator surface is adjacent to the proximal end of the actuated surface to a distal position where the actuator surface is adjacent to the distal end of the actuated surface.

25. The sub-assembly according to claim 22, wherein the sub-assembly comprises a delivery member cover axially movable relative to the housing, wherein the delivery member cover comprises a tubular section configured to surround a medicament delivery member of the medicament delivery device, wherein the delivery member cover comprises an arm extending from the tubular section towards the distal end of the housing, and wherein the actuator extends from the arm of the delivery member cover towards the longitudinal axis.

26. The sub-assembly according to claim 20, wherein the plunger rod is axially movable relative to the housing from a distal position where the proximally directed surface of the ledge of the plunger rod is spaced apart from the distally directed surface of the plunger rod holder to a proximal position where the proximally directed surface of the ledge of the plunger rod is adjacent to the distally directed surface of the plunger rod holder.

27. The sub-assembly according to claim 17, wherein the sub-assembly comprises an outer shell extending along the longitudinal axis between a proximal end and a distal end, wherein the outer shell is configured to receive a medicament container of the medicament delivery device, and wherein the housing is axially immovably arranged within the outer shell.

28. The sub-assembly according to claim 27, wherein the plunger rod comprises an engaging member, wherein the engaging member is closer to the distal end of the outer shell than the ledge of the plunger rod, wherein the housing comprises a counter engaging member engaged with the engaging member of the plunger rod, wherein one of the engaging member and the counter engaging member is arranged on a flexible arm, wherein the sub-assembly comprises a safety member removably attached to the distal end of the outer shell, wherein a cut-out arranged at the distal end of the outer shell, and wherein the safety member comprises a support wall protruding into the outer shell through the cut-out of the outer shell and configured to block the flexible arm from flexing.

29. The sub-assembly according to claim 28, wherein the engaging member of the plunger rod is a recess or cut-out is arranged in a sidewall of the plunger rod body, wherein the housing comprises a flexible arm extending from a distally directed wall of the housing towards a proximally directed wall of the outer shell, wherein the counter engaging member of the housing is a protrusion extending from the flexible arm, wherein the protrusion of the flexible arm protrudes into the recess or cut-out of the plunger rod body, and wherein the support wall of the safety member encloses an outer surface of the flexible arm of the housing.

30. The sub-assembly according to claim 28, wherein the cut-out of the outer shell is arranged in the proximally directed wall of the outer shell.

31. The sub-assembly according to claim 27, wherein the tubular section of the delivery member cover is telescopic relative to the proximal end of the outer shell.

32. The sub-assembly according to claim 17, wherein the biasing member is configured to bias the plunger rod relative to the housing in the direction of the longitudinal axis towards the proximal end of the housing.
